(19) United States
(12) Patent Application Publication
(10) Pub. No.: US 2007/0004942 A1
(43) Pub. Date: Jan. 4, 2007

(54) Method of preparation of an alkyne with an optically active hydroxyl group in the beta or gamma position of a triple bond and intermediates obtained

(76) Inventors: Laurent Garel, Lyon (FR); Thierry Schlama, Dardilly (FR)

Correspondence Address: STITES & HARBISON PLLC, 1199 NORTH FAIRFAX STREET, SUITE 900, ALEXANDRIA, VA, 22314, US

(21) Appl. No.: 11/476,196

(22) Filed: Jun. 28, 2006

(30) Foreign Application Priority Data
Jun. 30, 2005 (FR) 0506706

(51) Int. Cl. C07C 33/04; C07F 7/02

(52) U.S. Cl. 568/873; 556/466

(57) ABSTRACT

The present invention relates to a method of preparation of an alkyne with an optically active hydroxyl group in the β or γ position of a triple bond and intermediates obtained. The method of the invention for preparation of an alkyne with an optically active hydroxyl group in the β position of a triple bond is characterized in that it comprises the reaction, in the presence of a Lewis acid:
of a compound of formula (IV): 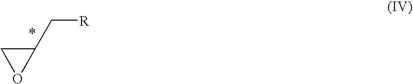 in which: R is a linear or branched alkyl group having from 1 to 6 carbon atoms. and of a compound of formula (V):
R′—C≡C-M (V) in which: R′ represents a hydrogen atom, a linear or branched alkyl group having from 1 to 8 carbon atoms, preferably a methyl group or a trialkylsilyl group. M represents a metal, preferably a metal of group (Ia) of the periodic table, preferably lithium. Another object of the invention comprises the production of an alkyne with an optically active hydroxyl group in the γ position of a triple bond by isomerization of an alkyne with an optically active hydroxyl group in the β position previously obtained.

The present invention relates to a method of preparation of an alkyne with an optically active hydroxyl group in the β or γ position of a triple bond and intermediates obtained.

Certain molecules especially in the pharmaceutical field contain a chiral synthon as shown below:

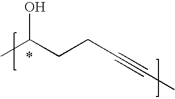

The difficulty in preparing molecules containing such synthons is connected with the fact that, as they are pharmaceutical molecules, very high chemical purity is required (generally greater than 95%) and that the presence of a hydroxyl group and of an unsaturation very easily leads to dimerized products, resulting in chemical contamination.

Moreover, the pharmaceutical market requires products that possess excellent optical purity, and the desired enantiomeric excess must preferably be greater than 99%.

Thus, the applicant proposes a method by which the aforesaid aims can be achieved, employing a novel intermediate.

The present invention relates to novel compounds corresponding to the following formula: 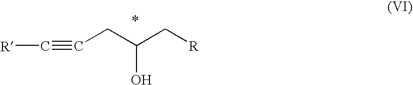
in which:
R represents a hydrogen atom or a linear or branched alkyl group having from 1 to 6 carbon atoms.
R′ represents a hydrogen atom, a linear or branched alkyl group having from 1 to 8 carbon atoms, preferably a methyl group or a trialkylsilyl group.

The preferred compounds correspond to general formula (VI) in which R is an alkyl group having from 2 to 5 carbon atoms, preferably an n-butyl group.

Regarding R′, X is preferably a hydrogen atom or a linear or branched alkyl group having from 1 to 8 carbon atoms.

R′ is preferably a methyl group.

Another object of the invention comprises the method of preparation of an alkyne with an optically active hydroxyl group in the e position of a triple bond, characterized in that it comprises the reaction, in the presence of a Lewis acid:
of a compound of formula (IV): 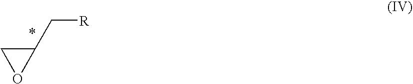
   in which:
R is a linear or branched alkyl group having from 1 to 6 carbon atoms.
and of a compound of formula (V):
   R′—C≡C-M (V)
   in which:
R′ represents a hydrogen atom, a linear or branched alkyl group having from 1 to 8 carbon atoms, preferably a methyl group or a trialkylsilyl group.
M represents a metal, preferably a metal of group (Ia) of the periodic table, preferably lithium.
leading to a compound of formula (VI): 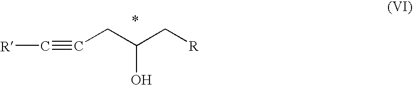
in which R and R′ have the meaning given previously.

The invention also relates to the use of the compounds of formula (VI) for the preparation of an alkyne with an optically active hydroxyl group in the γ position of a triple bond.

According to another object of the invention, a method has been found for the preparation of an alkyne with an optically active hydroxyl group in the γ position of a triple bond, characterized in that it comprises the reaction of isomerization, in the presence of a superbase of the metal amide type, of the compound of formula (VI) in which R′ represents a linear or branched alkyl group having from 1 to 8 carbon atoms.

According to the method of the invention, a compound of formula (VI) is prepared by reacting a compound of formula (IV) namely a chiral epoxyalkane with a lithium alkynide of formula (VI), with the reaction taking place in the presence of a Lewis acid.

According to a preferred embodiment of the invention, first a chiral epoxyalkane of formula (IV) is prepared according to a method comprising:
(1) the reaction in the presence of a copper catalyst:
   of a chiral haloepoxide represented by the following formula: 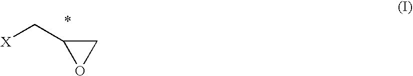
      where X represents a leaving group, preferably a halogen atom or a sulphonate group of formula —OSO₂—R₁, in which R₁ is a hydrocarbon group.
and of an aliphatic organometallic compound corresponding to the formula:
   R—Y (II)
   in which:
R is a linear or branched alkyl group having from 1 to 6 carbon atoms.
Y represents:
   .MgX², in which X² represents a halogen atom.
   .Li⁺, Na⁺.
      leading to a compound of formula (III): 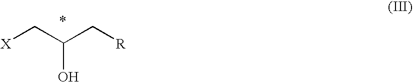
      where X and R have the meanings given previously.
(2) followed by a reaction of nucleophilic substitution by reacting the compound of formula (III) with a base, which then leads to the compound of formula (IV).
   Reaction of a Haloepoxide and an Organometallic Compound.

According to the method of the invention, we start from a chiral haloepoxide which can be represented by the following formula: 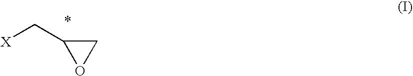
where X represents a leaving group, preferably a halogen atom or a sulphonate group of formula —OSO₂—R₁, in which R₁ is a hydrocarbon group.

In formula (I), X represents a halogen atom selected from chlorine, bromine and iodine, preferably a chlorine atom.

In the formula of the sulphonate group, R₁ is a hydrocarbon group of any kind. However, since X is a leaving group, it is economically beneficial if R₁ is of a simple nature, and represents more particularly a linear or branched alkyl group having from 1 to 4 carbon atoms, preferably a methyl or ethyl group but it can also represent for example a phenyl or tolyl group or a trifluoromethyl group.

Preferably a chlorine atom is selected, as preferred leaving groups.

According to the invention, the compound of formula (I) reacts with an aliphatic organometallic compound corresponding to the formula:
R—Y (II)
in which:
R is a linear or branched alkyl group having from 1 to 6 carbon atoms.
Y represents:
   .MgX², in which X² represents a halogen atom.
   .Li⁺, Na⁺.

The compound of formula (II) employed more particularly corresponds to formula (II) in which R is a linear and branched alkyl group having from 1 to 6 carbon atoms, preferably 2 to 5 carbon atoms and more particularly the methyl, ethyl, propyl, isopropyl, n-butyl, isobutyl, sec-butyl, tert-butyl, n-pentyl, isopentyl, n-hexyl groups.

The n-butyl group is preferred.

With regard to the definition of Y, it is preferably an organomagnesium compound, preferably an organobromo- or organochloromagnesium compound. Thus, in formula (II), a bromine or chlorine atom is preferably selected, as preferred groups X².

Organobromo- or organochloromagnesium compounds, for example methylmagnesium chloride or ethylmagnesium chloride are available commercially, but they can also be prepared according to the teaching of the prior art (J. Org. Chem., 2000, 65(7), 2231).

The amount of the reactants employed is such that the ratio of the number of moles of aliphatic organometallic compound of formula (II) to the number of moles of haloepoxide of formula (I) is preferably greater than or equal to 1, preferably between 1 and 1.5, and more preferably between 1 and 1.2.

The reaction of the haloepoxide and of the aliphatic organometallic compound takes place in the presence of a coupling catalyst which is preferably copper catalyst.

As examples of catalysts that can be employed, we may mention the organic or inorganic compounds of copper(I) or of copper(II).

The catalysts employed in the method of the invention are known products.

As examples of catalysts of the invention, we may mention notably as copper compounds, cuprous bromide, cupric bromide, cuprous iodide, cuprous chloride, cupric chloride.

The halides of copper, preferably of copper(I), are preferred.

Preferably, cuprous iodide is selected.

The amount of catalyst employed expressed by the molar ratio of the number of moles of catalyst to the number of moles of compound of formula (I) generally varies between 0.05 and 0.2, preferably between 0.1 and 0.15.

The reaction temperature is advantageously between −78° C. and −40° C., and preferably between −65° C. and −50° C.

Generally, the reaction is carried out under autogenous pressure of the reactants.

According to a preferred variant of the method of the invention, the method of the invention is carried out under a controlled atmosphere of inert gases. An atmosphere of rare gases, preferably argon, can be established, but it is more economical to use nitrogen.

The method according to the invention is carried out in the liquid phase.

As the magnesium reactant is available commercially in solution in an organic solvent, the reaction according to the invention is carried out in the presence of the solvent generally employed for its synthesis.

As examples of organic solvents, we may mention among others, solvents of the ether type such as the aliphatic, cycloaliphatic or aromatic ether oxides, more particularly ethyl ether, dioxan, tetrahydrofuran, preferably tetrahydrofuran.

It is possible for there to be a co-solvent and we may mention more particularly the aliphatic, cycloaliphatic or aromatic hydrocarbons, preferably hexane, methylcyclohexane, toluene, xylenes. Toluene is the preferred solvent.

It should be noted that the amount of solvent of the ether type is predominant, as it can vary between 50 and 100% of the total weight of the mixture comprising the solvent of the ether type and the co-solvent.

The concentration of the compound of formula (I) employed in the solvent an vary between 0.5 and 2 mol/l.

From a practical standpoint, the method is simple to carry out.

The haloepoxide of formula (I), organic solvent and catalyst are mixed together at low temperature, as previously defined.

The aliphatic organometallic compound (II), preferably alkylmagnesium halide in solution in a solvent, is added progressively, preferably continuously. The reverse can also be done.

Stirring is continued until the reactants have been consumed completely, which can be monitored by an analytical method, for example gas chromatography.

At the end of the reaction, the reaction is stopped by adding a saturated salt solution, preferably a solution of an alkali metal hydrogencarbonate, preferably of sodium or potassium, or an ammonium halide, preferably ammonium chloride. Preference is given to the latter.

The compound corresponding to the following formula is obtained: 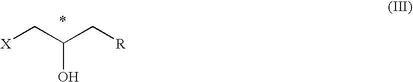
where R and X have the meanings given previously.

The compound of formula (III) is isolated in a conventional manner.

For example, the compound of formula (III) can be extracted in an organic solvent, which is insoluble in water and which dissolves it.

As preferred examples of solvents, we may mention a solvent of the ester type, preferably ethyl acetate, or a solvent of the ether type, preferably methyl tert.-butylether.

The aqueous and organic phases are separated.

The organic phase comprises the compound of formula (III) and the aqueous phase contains various salts.

The aqueous and organic phases are separated and the organic phase is washed with a basic solution, preferably an aqueous solution of soda (for example with a concentration from 15 to 30 wt. %), until the reaction is neutral.

The compound of formula (III) is recovered from this organic phase by conventional means.

Preferably drying of the organic phase is carried out, for example by means of magnesium sulphate or sodium sulphate, then the organic phase is concentrated by distillation of the solvent, most often under reduced pressure.

Preparation of the Chiral Epoxyalkane.

In the next stage, the chiral epoxyalkane of formula (IV) is prepared according to a reaction of nucleophilic substitution, by reacting the compound of formula (III) with a base.

Among the bases that can be used, we may mention among others, inorganic bases such as carbonates, hydrogencarbonates or hydroxides of alkali metals, preferably of sodium, of potassium, of caesium or of alkaline-earth metals, preferably of calcium, barium or magnesium.

It is also possible to use hydrides of alkali metals, preferably sodium hydride or alcoholates of alkali metals, preferably of sodium or of potassium, and more preferably sodium methylate, ethylate or tert.-butylate.

The inorganic base is used advantageously in the form of a solid.

Organic bases are also suitable, such as DBU (1,8-diazabicyclo[5.4.0]undec-7-ene), DBN (1,5-diazabicyclo[4.3.0]non-5-ene) and DABCO (1,4-diazabicyclo[2.2.2]octane).

Among bases, preferably hydroxides of alkali metals, preferably sodium hydroxide, are selected.

The amount of base used is such that the ratio of the number of moles of base to the number of moles of the compound of formula (III) varies between 1 and 3, and is preferably equal to about 2.

The reaction of nucleophilic substitution is carried out in the presence of an organic solvent.

A solvent is selected which is inert in the reaction conditions.

As more specific examples of solvents suitable for the present invention, we may mention preferably polar aprotic solvents such as dimethylsulphoxide, sulpholane.

As other examples of less-polar organic solvents suitable for the invention, we may mention notably aliphatic, cycloaliphatic or aromatic halogenated or unhalogenated hydrocarbons; ether-oxides.

It is also possible to use a solvent of the alcohol type. We may mention quite particularly methanol, ethanol, n-propanol, isopropanol, cyclohexanol.

As examples of solvents, we may mention aliphatic, cycloaliphatic or aromatic ether-oxides and, more particularly, diethyl oxide, dipropyl oxide, diisopropyl oxide, dibutyl oxide, methyl tert.-butylether, dipentyl oxide, diisopentyl oxide, ethyleneglycol dimethylether (or 1,2-dimethoxyethane), diethyleneglycol dimethylether (or 1,5-dimethoxy-3-oxapentane), dioxan, tetrahydrofuran.

Tetrahydrofuran is selected advantageously.

A mixture of organic solvents can also be used.

The amount of organic solvent used is preferably selected so that the concentration by weight of the starting substrate in the solvent is between 5 and 40%, preferably between 10 and 20%.

A mixture of solvents can also be used.

The amount of organic solvent to use is determined in relation to the nature of the organic solvent selected.

It is determined in such a way that the concentration of the compound of formula (III) is preferably between 1 and 3 mol/l.

The reaction of nucleophilic substitution takes place at a temperature which is advantageously between 10° C. and 50° C., and preferably at room temperature, which is generally between 15° C. and 25° C.

Said reaction is carried out at atmospheric pressure.

From the practical standpoint, the compound of formula (III) is dissolved in the organic solvent and the base is added.

Stirring is maintained, preferably at room temperature, for one or two hours.

The compound corresponding to the following formula is obtained: 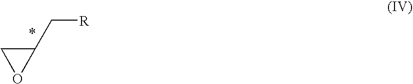
where R has the meaning given previously.

The chiral epoxide is recovered by conventional means.

For example, the base can be separated using the conventional techniques of solid/liquid separation, for example by filtration on Celite, then distillation is performed under reduced pressure, preferably 50 or 60 mm of mercury.

Condensation of the Metal Alkynide and Epoxyalkane.

According to the method of the invention, in this stage, the epoxyalkane of formula (IV) is reacted, in the presence of a Lewis acid, with a metal alkynide of formula (V):
R′—C≡C-M (V)
in which:
R′ represents a hydrogen atom, a linear or branched alkyl group having from 1 to 8 carbon atoms, preferably a methyl group or a trialkylsilyl group,
M represents a metal, preferably a metal of group (Ia) of the periodic table, preferably lithium.

The method of the invention involves a compound of formula (V) in which R′ represents a hydrogen atom, a linear or branched alkyl group having from 1 to 8 carbon atoms or a trialkylsilyl group.

R′ preferably represents a hydrogen atom or a methyl group.

Note that in the present text, “trialkysilyl group” means a group of the type —Si—(R₂)₃ in which R₂ is a linear or branched alkyl group preferably having from 1 to 4 carbon atoms, preferably a methyl group.

With regard to M, M represents a monovalent metal, preferably a metal of group (Ia) of the periodic table of the elements and more particularly lithium, sodium or potassium.

M is preferably lithium.

Reference may be made to the periodic table of the elements published in the Bulletin de la Société Chimique de France No. 1 (1966).

As more particular examples of alkynides used, we may mention quite especially lithium acetylide or lithium propynide.

A lithium alkynide which is prepared in a conventional manner is used.

The alkyne is reacted in solution in an organic solvent, preferably tetrahydrofuran, with an alkyllithium in solution in an organic solvent, preferably an aliphatic hydrocarbon, and preferably hexane, at a temperature between −78° C. and −20° C.

A suspension of metal alkynide is generally obtained.

Then said alkynide is reacted with the chiral epoxide of formula (IV), in the presence of a Lewis acid.

The amount of the reactants employed is such that the molar ratio metal alkynide/chiral epoxide is advantageously between 1 and 2.

The method of the invention involves the use of a Lewis acid.

By “Lewis acid”, we mean an entity that is capable of accepting an electron doublet. Every Lewis acid has an electron vacancy.

As examples of Lewis acids, we may mention trimethylaluminium, trimethylgallium, aluminium diethylchloride, gallium aluminate, boron trifluoride.

As for the source of boron trifluoride, it is possible to use BF₃ in the form of a gas.

However, it is preferable to use complexes of boron trifluoride containing between about 20 and 70 wt. % of boron trifluoride.

As examples of complexes, we may mention in particular complexes comprising boron trifluoride combined with an organic compound of the Lewis base type, selected from water, ethers, alcohols and phenols, acetic acid, acetonitrile.

As examples of ethers, we may mention notably dimethyl ether, diethyl ether, dibutyl ether, methyl-tert-butylether, tetrahydrofuran.

As other solvents, we may mention among others, alcohols such as methanol, propanol or phenol.

Sources of boron trifluoride which are commercially available are preferably used.

We may mention notably complexes of BF₃, 2 H₂O, of BF₃ and acetic acid, diethyl ether, dibutyl ether or methyl-tert-butylether.

As preferred reactants, preferably boron trifluoride is selected, together with water, acetic acid or diethyl ether.

The amount of Lewis acid used is such that the molar ratio Lewis acid/compound of formula (IV) varies between 1 and 2, preferably between 1.0 and 1.5.

According to the method of the invention, the reaction is carried out in an organic environment, which means that an organic solvent or optionally a mixture of organic solvents is present.

An aprotic, polar or apolar solvent is used.

As non-limiting examples of solvents suitable for the method of the invention, we may mention the ethers as previously mentioned.

Out of all these solvents, tetrahydrofuran is preferred.

The reaction of condensation takes place at a temperature between −78° C. and −20° C.

From a practical standpoint, the reactants can be used in any order.

A preferred embodiment comprises progressively adding the Lewis acid, then the chiral epoxyalkane in the reaction mixture comprising the metal alkynide of formula (V).

Another embodiment comprises progressively adding the chiral epoxyalkane then the Lewis acid in the reaction mixture comprising the metal alkynide of formula (V).

The reaction mixture is maintained at the temperature previously defined for 2 to 8 hours.

The reaction is continued until the chiral epoxyalkane disappears completely, monitored by gas chromatography.

At the end of reaction, the reaction is stopped by adding a saturated salt solution, preferably a solution of a hydrogencarbonate of an alkali metal, preferably sodium or potassium, or an ammonium halide, preferably ammonium chloride. Ammonium chloride is preferred.

The compound corresponding to the following formula is obtained: 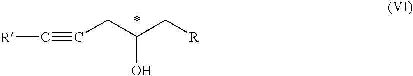
where R and R′ have the meaning given previously.

The compound of formula (VI) is separated in a conventional manner.

For example, the compound of formula (VI) can be extracted in an organic solvent, which is insoluble in water and which dissolves it.

As preferred examples of solvents, we may mention a solvent of the ester type, preferably ethyl acetate or a solvent of the ether type, preferably methyl-tert.-butylether.

The aqueous and organic phases are separated.

The organic phase contains the compound of formula (VI) and the aqueous phase contains the various salts.

The aqueous and organic phases are separated and the organic phase is washed with a saturated solution of sodium chloride.

The compound of formula (VI) is recovered conventionally, from this organic phase.

Drying of the organic phase is preferably carried out, for example by means of magnesium sulphate or sodium sulphate, then the organic phase is concentrated by distillation of the solvent, generally under reduced pressure.

The product can also be recovered by chromatography on silica.

It is obtained in the form of an oil.

Reaction of Isomerization.

According to the present invention, an alkyne with an optically active hydroxyl group in the y position with respect to the triple bond is prepared according to a reaction of isomerization of an alkyne with an optically active hydroxyl group in the β position with respect to the triple bond.

Thus, another object of the present invention is a method by which an optically active hydroxyl group is introduced in the γ position of a triple bond, characterized in that it comprises the reaction of isomerization, in the presence of a superbase of the metal amide type, of the compound of formula (VI) in which R′ is a linear or branched alkyl group having from 1 to 8 carbon atoms.

One embodiment of the reaction of isomerization of the compound of formula (VI) comprises bringing the latter into contact with a superbase of the metal amide type.

A first method of production comprises reacting a hydride of an alkali metal preferably sodium and potassium with a diaminoalkane, preferably 1,2-diaminoethane, 1,3diaminopropane.

We may mention potassium 3-aminopropylamide or KAPA.

The diaminoalkane is used in excess.

The amounts of each reactant are such that the ratio of diaminoalkane to metal hydride varies between 1 and 10.

Another type of reactant that may be suitable for carrying out the isomerization of the triple bond results from the addition of a metal alcoholate, preferably of an alkali metal, to a lithium salt of a diaminoalkane.

As examples of alcoholates of alkali metals, preferably of sodium or of potassium, we may mention more preferably methylate, ethylate or tert.-butylate of sodium or of potassium. Potassium tert.-butylate is preferred.

As for the diaminoalkane, 1,2-diaminoethane or 1,3-diaminopropane is selected advantageously.

The amount of lithium used is generally selected in such a way that the molar ratio of the lithium to the compound of formula (VI) varies between 1 and 6.

The amount of metal alcoholate used is generally selected in such a way that the molar ratio of metal alcoholate to the compound of formula (VI) varies between 1 and 10, preferably around 6.

The amount of diaminoalkane employed is most often such that the molar ratio of diaminoalkane to the compound of formula (VI) varies between 30 and 50.

According to a preferred embodiment of the invention, mixing of the lithium and diaminoalkane is carried out at room temperature.

The reaction mixture is heated to a temperature in the range from 50° C. to 70° C.

A white suspension appears.

It is cooled to room temperature and the metal alcoholate is added.

The reaction mixture is stirred for between 15 min and 30 min.

The alkyne of formula (VI) is then added.

The reaction mixture is stirred at a temperature between 0° C. and 25° C. for between 15 min and 24 hours, preferably for about 30 min.

The reaction is stopped by bringing the reaction mixture into contact with ice water.

An alkyne with an optically active hydroxyl group in the γ position of the triple bond is obtained.

When the starting alkyne is a compound of formula (VI) in which R′ is a methyl group, an alkyne with an optically active hydroxyl group in the γ position of the triple bond is obtained with the following formula: 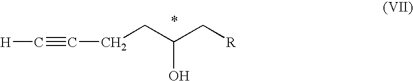
where R has the meaning given previously.

The alkyne obtained is isolated by conventional means.

For example, the alkyne obtained can be extracted in an organic solvent, which is insoluble in water and which dissolves it.

As preferred examples of solvents, we may mention a solvent of the ether type, preferably methyltert.-butylether.

The aqueous and organic phases are separated.

The organic phase contains the alkyne with an optically active hydroxyl group in the γ position of the triple bond and the aqueous phase contains the various salts.

The aqueous and organic phases are separated and the organic phase is washed with an aqueous solution of acid (for example 10% HCI) then a saturated solution of sodium chloride.

The alkyne with an optically active hydroxyl group in the γ position of the triple bond is recovered in a conventional manner from this organic phase.

Preferably drying of the organic phase is carried out, for example by means of magnesium sulphate or sodium sulphate, then the organic phase is concentrated by solvent distillation, generally under reduced pressure.

The method of the invention makes it possible to obtain an alkyne with an optically active hydroxyl group in the γ position of a triple bond that meets the requirements of chemical and enantiomeric purity.

Examples of carrying out the invention are given below for illustration and they are not limiting.

In the examples, “yield” means the ratio of the number of moles of product formed to the number of moles of substrate used.

### EXAMPLE 1

### Synthesis of (S)-1-chloro-2-heptanol

A small amount of iodine is added to a suspension of magnesium (47.52 g, 1.98 mol) in THF (350 ml) to activate the magnesium, and a solution of 1-chlorobutane (167 g, 188 ml, 1.8 mol) in THF (370 ml) is added dropwise with gentle heating.

After addition, the mixture is heated under reflux for 30 min and cooled to room temperature.

The solution is approx. 2.0 M of n-butylmagnesium chloride.

A solution of n-butylmagnesium chloride (0.9 L, 2.0 M, 1.8 mol) in THF is added slowly, over 2.5 h, to a mixture of (S)-epichlorohydrin (139 g, 1.5 mol) of Cul (28.58 g, 0.15 moles) in THF (750 ml) at −70/−60° C. cooled with a dry ice-acetone mixture) in a 3-litre three-necked flask, under a nitrogen atmosphere.

The mixture is stirred at γ65° C./−50° C. for more than an hour.

A saturated solution of ammonium chloride is added at −50° C.

The mixture is separated and the aqueous layer is extracted with ethyl acetate (2×600 ml) (if a solid is present in the aqueous layer, the solid is removed by filtration and then washed with 50 ml of ethyl acetate).

The organic phases are combined and washed respectively with 100 ml of a 15% ammonia solution (once or twice) and with a saturated solution of sodium chloride (once).

The organic phase is dried over sodium sulphate and concentrated to give a yellow liquid (220 g). The yield is 85%.

This liquid is used as it is in the next stage without further purification.

### EXAMPLE 2

### Synthesis of 1,2-(S)-epoxyheptane

Pulverized soda (120 g) is added to a solution of 1-chloro-2-heptanol (220 g).

The mixture is stirred vigorously at room temperature for 2 hours and then filtered on Celite and washed with THF.

The filtrate is concentrated at 38° C. in a rotary evaporator to give a brown or yellow liquid (109 g).

The yield obtained is 68%.

The product is purified by distillation under reduced pressure of 50-60 mm of mercury to give 75.1 g of a colourless liquid, which corresponds to a yield of 51%.

The enantiomeric excess is determined by GC for the chiral phase and is greater than 99% [of enantiomer (S)].

### EXAMPLE 3

### Synthesis of (S)-dec2-yn-5-ol

Propyne (2.2 mL, 35.1 mmol) is condensed in THF (23.2 mL) at −78° C. then fed into a 100-mL three-necked reactor equipped with a temperature probe, a nitrogen inlet pipe, a stirrer and a dry ice/acetone bath.

The solution of propyne in THF is treated, while stirring, with n-butyllithium 2.5 M (in hexane) (4.77 g, 17.5 mmol) added slowly so as not to exceed −50° C.

As the n-butyllithium is added, formation of lithium propynide is noted from the appearance of a characteristic white suspension (Encyclopedia of reagents for organic synthesis, editor: L. A. Paquette, Wiley, 1995, page 4339).

This white suspension is left for half an hour, then boron trifluoride etherate BF₃.Et₂O (1.31 g, 9.2 mmol) is added in a few seconds.

After 15 min, 1,2(S)epoxyheptane (1 g, 8.8 mmol) in solution in THF (1 mL) is added at −78° C.

Once again, care is taken not to exceed −50° C.

The progress of the reaction is monitored by gas chromatography (GC) and thin-layer chromatography (TLC) until 1,2(S)-epoxyheptane is no longer observed.

The solution is then treated with a saturated aqueous solution of ammonium chloride NH₄Cl (31.9 g) added at −78° C. and the aqueous and organic phases are separated.

The aqueous phase is extracted 3 times with methyl-tert-butyl ether (3×30 mL) and the organic extracts are combined and washed with a saturated aqueous solution of sodium chloride (2×20 mL).

The organic phase is dried over sodium sulphate and then concentrated under vacuum (40° C., 500 mbar).

1 g of a light yellow oil is obtained, which is found to be (S)-dec-2-yn-5-ol, which is determined by GC.

The yield is found to be 55% and the purity of the raw reaction product is 75% w/w.

### EXAMPLE 4

### Synthesis of (S)-dec-1-yn-5-ol

Lithium (0.56 g, 81.2 mmol), cut into small pieces from a lithium wire and washed in ether and then 1,3-diaminopropane (36.1 g, 48.7 mmol), is placed in a 50-mL three-necked reactor equipped with a condenser and a magnetic stirrer.

The stirred solution turns a very dark blue, and release of gas and exothermic character are noted.

The solution is heated at 70° C. for 2 hours and turns white over time (suspension).

On cooling to room temperature (i.e. 20° C.), potassium tert-butylate ^{t}BuOK (6.39 g, 54.1 mmol) is added.

The solution turns yellow and then orange.

The exothermic character is noted, and stirring is continued at room temperature for 20 minutes.

Then 2.22 g of (S)-dec-2-yn-5-ol (94% w/w, 13.5 mmol) is added, monitoring the reaction by GC.

The reaction mixture is poured into 85 g of ice water with stirring (exothermic character).

The aqueous and organic phases are separated.

The aqueous phase is extracted 3 times with diethyl ether (3×50 mL) and the organic extracts are combined and washed with water (50 mL), with a 10% aqueous solution of HCl (40 mL) then with a saturated aqueous solution of sodium chloride (2×40 mL).

The organic phase is then dried over sodium sulphate and concentrated under vacuum.

1.4 g of a light yellow oil is obtained, which is found to be (S)-dec-2-yn-5-ol, which is determined by GC.

The yield is found to be 66% and the purity of the raw reaction product is 96% w/w. The enantiomeric excess is determined by GC on the chiral phase and is greater than 99% [of enantiomer (S)].

1. Compound of formula (VI) corresponding to the following formula: 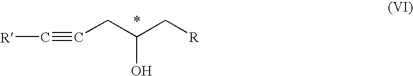
in which:
R represents a hydrogen atom or a linear or branched alkyl group having from 1 to 6 carbon atoms,
R′ represents a hydrogen atom, a linear or branched alkyl group having from 1 to 8 carbon atoms, preferably a methyl group or a trialkylsilyl group.

2. Compound according to claim 1, wherein it corresponds to formula (VI) in which R is an alkyl group having from 2 to 5 carbon atoms, preferably an n-butyl group.

3. Compound according to claim 1, wherein it corresponds to formula (VI) in which R′ is a hydrogen atom.

4. Compound according to claim 1, wherein it corresponds to formula (VI) in which R′ is a linear or branched alkyl group having from 1 to 8 carbon atoms, preferably a methyl group.

5. Method of preparation of an alkyne with an optically active hydroxyl group in the β position of a triple bond, comprising the reaction, in the presence of a Lewis acid:
of a compound of formula (IV): 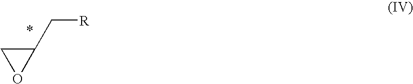
in which:
R is a linear or branched alkyl group having from 1 to 6 carbon atoms.
and of a compound of formula (V):
R′—C≡C-M (V)
in which:
R′ represents a hydrogen atom, a linear or branched alkyl group having from 1 to 8 carbon atoms, preferably a methyl group or a trialkylsilyl group,
M represents a metal, preferably a metal of group (Ia) of the periodic table, preferably lithium.
leading to a compound of formula (VI): 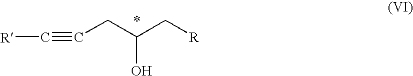
where R and R′ have the meaning given previously.

6. Method according to claim 5, wherein the chiral epoxyalkane corresponds to formula (IV) in which R is a linear and branched alkyl group having from 1 to 6 carbon atoms, preferably 2 to 5 carbon atoms and preferably an n-butyl group.

7. Method according to claim 5, wherein the chiral epoxyalkane corresponds to formula (IV) in which R is an alkyl group.

8. Method according to claim 5, wherein the metal alkynide corresponds to formula (V) in which M represents lithium.

9. Method according to claim 5, wherein the metal alkynide corresponds to formula (V) in which R′ represents a hydrogen atom or a methyl group.

10. Method according to claim 8, wherein the metal alkynide of formula (V) is lithium acetylide or lithium propynide.

11. Method according to claim 5, wherein the Lewis acid is trimethylaluminium, trimethylgallium, aluminium diethylchloride, gallium aluminate, boron trifluoride.

12. Method according to claim 11, wherein the Lewis acid is a complex of BF₃, 2 H₂O, or of BF₃ and of acetic acid, of diethyl ether, of dibutyl ether or of methyl-tert-butylether.

13. Method according to claim 5, wherein the reaction of condensation takes place at a temperature between −78° C. and −20° C.

14. Method according to claim 5, wherein the compound of formula (IV) is obtained by a reaction of nucleophilic substitution, in the presence of a base of the compound of formula (III): 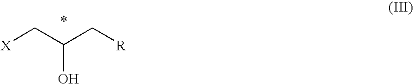
where R represents a hydrogen atom, a linear or branched alkyl group having from 1 to 8 carbon atoms, preferably a methyl group or a trialkylsilyl group.

15. Method according to claim 14, wherein the base is an inorganic base such as a carbonate, hydrogencarbonate or hydroxide of an alkali metal, preferably of sodium, of potassium, of caesium or of an alkaline-earth metal, preferably of calcium, barium or magnesium: an alkali metal hydride, preferably sodium hydride; an alkali metal alcoholate, preferably of sodium or of potassium, and more preferably sodium methylate, ethylate or tert.-butylate; an organic base such as DBU (1,8-diazabicyclo[5.4.0]undec-7-ene), DBN (1,5-diazabicyclo[4.3.0]non-5-ene) and DABCO (1,4-diazabicyclo[2.2.2]octane.

16. Method according to claim 14, wherein the base is soda.

17. Method according to claim 14, wherein the reaction of nucleophilic substitution is carried out in the presence of an organic solvent, preferably polar and aprotic, which is preferably selected from methylsulphoxide, sulpholane, halogenated or unhalogenated aliphatic, cycloaliphatic or aromatic hydrocarbons; ether-oxides; alcohols.

18. Method according to claim 17, wherein the organic solvent is selected from diethyl oxide, dipropyl oxide, diisopropyl oxide, dibutyl oxide, methyltert.-butylether, dipentyl oxide, diisopentyl oxide, ethyleneglycol dimethylether (or 1,2-dimethoxyethane), diethyleneglycol dimethylether (or 1,5-dimethoxy-3-oxapentane), dioxan, tetrahydrofuran and preferably tetrahydrofuran.

19. Method according to claim 14, wherein the reaction of nucleophilic substitution takes place at a temperature between 10° C. and 50° C., preferably at room temperature.

20. Method according to claim 14, wherein the compound of formula (III) is obtained by the reaction in the presence of a copper catalyst:
of a chiral haloepoxide represented by the following formula: 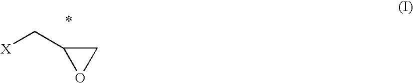
where X represents a leaving group, preferably a halogen atom or a sulphonate group of formula —OSO₂—R₁, in which R₁ is a hydrocarbon group,
and of an aliphatic organometallic compound corresponding to the formula:
R—Y (II)
in which:
R is a linear or branched alkyl group having from 1 to 6 carbon atoms.
Y represents:
.MgX², in which X² represents a halogen atom.
.Li+, Na+.

21. Method according to claim 20, wherein the copper catalyst is a copper halide, preferably a halide of copper(I) and preferably cuprous iodide.

22. Method according to claim 20, wherein the reaction temperature is between −78° C. and −40° C., and preferably between −65° C. and −50° C.

23. Method according to claim 20, wherein the reaction is carried out in an organic solvent, preferably a solvent of the ether type, an aliphatic, cycloaliphatic or aromatic hydrocarbon.

24. Method according to claim 23, wherein the organic solvent is the organic solvent of the magnesium reactant, preferably tetrahydrofuran.

25. Method of introducing an optically active hydroxyl group in the γ position of a triple bond, comprising the reaction of isomerization, in the presence of a superbase of the metal amide type, of the compound of formula (VI) in which R′ is a linear or branched alkyl group having from 1 to 8 carbon atoms.

26. Method according to claim 25, wherein the reaction of isomerization is carried out in the presence of a superbase obtained by reaction of a hydride of an alkali metal preferably sodium and potassium with a diaminoalkane, preferably 1,2-diaminoethane, 1,3-diaminopropane.

27. Method according to claim 25, wherein the reaction of isomerization is carried out in the presence of a superbase obtained by addition of a metal alcoholate, preferably of an alkali metal, to a lithium salt of a diaminoalkane.

28. Method according to claim 25, wherein the reaction of isomerization takes place between 0° C. and 25° C.
